(19) United States
(12) Patent Application Publication
(10) Pub. No.: US 2019/0388098 A1
(43) Pub. Date: Dec. 26, 2019

(54) Training System for Tourniquet Application

(71) Applicant: Charles River analytics, Inc., Cambridge, MA (US)

(72) Inventors: Benjamin Bauchwitz, Cambridge, MA (US); James M. Niehaus, Apex, NC (US); Peter Weyhrauch, Newton, MA (US); Noa Palmon, Cambridge, MA (US); Carla Pugh, Madison, WI (US)

(21) Appl. No.: 16/447,266

(22) Filed: Jun. 20, 2019

Related U.S. Application Data
(60) Provisional application No. 62/687,491, filed on Jun. 20, 2018.

(51) Int. Cl. A61B 17/132; A61B 5/00; G09B 23/30

(52) U.S. Cl. CPC A61B 17/132; A61B 5/026; G09B 23/30; A61B 5/0053

(57) ABSTRACT

Systems and methods are described which provide a combination of sensors and software to enhance the process of learning how to apply a junctional tourniquet to non-compressible hemorrhages, using a separate force sensing system, providing detailed feedback on the physical process of how the tourniquet was applied, and offering novel flexibility in application and sensing.

### CROSS-REFERENCE TO RELATED APPLICATION

This application is based upon and claims priority to U.S. provisional patent application 62/687,491, entitled “Training System for Tourniquet Application,” filed 20 Jun. 2018, attorney docket number 243012-0017R; the entire content of this noted provisional application is incorporated herein by reference.

### STATEMENT REGARDING FEDERALLY SPONSORED RESEARCH

This invention was made with government support under contract numbers Wx81XWH-17-C-0098 and W81XWH-13-C-0021 awarded by U.S. Army Medical Research and Material Command. The government has certain rights in the invention.

### BACKGROUND

Battlefield medical treatment has advanced tremendously in recent years, exemplified by the development of Tactical Combat Casualty Care (TCCC), which emphasizes immediate attention to life-threatening conditions, such as airway blockage or fatal bleeding. Advances in practice and technology, such as QuikClot Combat Gauze™ and traditional tourniquets on limbs, have prevented many combat-related deaths.

Unfortunately, certain injuries, such as non-compressible hemorrhages in junctional or inguinal areas (such as pelvis, groin, underarms, or neck), cannot be addressed by these techniques. Inventors have developed new types of junctional tourniquets, also referred to as junctional tourniquet devices, to respond to this challenge. Examples of junctional tourniquet devices include the Abdominal Aortic Junctional Tourniquet (“AAJT”), the SAM Junctional Tourniquet (“SJT”), the Junctional Emergency Treatment Tool (“JETT”), and the Combat Ready Clamp (“CRoC”), which address abdominal and pelvic injuries.

While these hemorrhage-control technologies have been validated and approved for use by the FDA, as well as accepted into the TCCC guidelines, training systems for these technologies have not been fully realized.

Traditional methods of teaching use of tourniquets include practicing placing the tourniquet on a live human (providing no objective metrics, and cannot use full pressure); practicing placing the tourniquet on an animal (providing no objective metrics, are costly, and may raise ethical questions); and, practicing placing the tourniquet on a manikin (proving no objective metrics, and no feedback on performance).

### SUMMARY

An aspect of the present disclosure is directed to systems and methods providing a combination of sensors and software to enhance the process of learning how to apply a junctional tourniquet to non-compressible hemorrhages, using a separate force sensing system, providing detailed feedback on the physical process of how the tourniquet was applied, and offering novel flexibility in application and sensing.

These, as well as other components, steps, features, objects, benefits, and advantages, will now become clear from a review of the following detailed description of illustrative embodiments, the accompanying drawings, and the claims.

### BRIEF DESCRIPTION OF DRAWINGS

The drawings are of illustrative embodiments. They do not illustrate all embodiments. Other embodiments may be used in addition or instead. Details that may be apparent or unnecessary may be omitted to save space or for more effective illustration. Some embodiments may be practiced with additional components or steps and/or without all of the components or steps that are illustrated. When the same numeral appears in different drawings, it refers to the same or like components or steps,

FIG. 1 depicts an example a tourniquet master training (“TMT”) system, in accordance with an exemplary embodiment of the present disclosure.

FIG. 2 depicts different junctional tourniquets, and includes views (A)-(B), with (A) showing an abdominal aortic junctional tourniquet (“AAJT”), and (B) showing a combat ready clamp (“CRoC”).

FIG. 3 depicts a diagram of an example of a message queue serving to coordinate and organize input from multiple sources and allow it to be queried by and submitted to software applications, in accordance with an exemplary prototype a TMT system according to the present disclosure.

FIG. 4 depicts hardware for an exemplary embodiment of a TMT sensor setup.

FIG. 5 depicts a curve used to convert sensor voltages to force data for addressing voltage saturation and to distinguish higher force values applied to the sensors, in accordance with an exemplary embodiment of the present disclosure.

FIG. 6 depicts sensor data for AAJT application with phases of the tourniquet application process marked, in accordance with an exemplary embodiment of the present disclosure.

FIG. 7 depicts a system diagram and training algorithm for a TMT virtual mentor, in accordance with exemplary embodiments of the present disclosure.

FIG. 8 depicts a trainee display for a TMT virtual mentor, showing feedback on three critical metrics: location, pressure, and time to complete, in accordance with an exemplary embodiment of the present disclosure.

FIG. 9 depicts a trainee display for a TMT virtual mentor, showing the outcome of a CRoC being applied to the correct location at the correct pressure; the pulse display at the right shows a flat line, indicating that blood flow has stopped.

FIG. 10 depicts nine pressure templates used to determine location metrics in accordance with an exemplary embodiment of the present disclosure; each template corresponds to a tourniquet being at a particular location, and records what the sensor data look like when the tourniquet is at that location.

FIG. 11 depicts an example of an instructor display showing the sensors (larger shaded squares, some with split shading) and the pressure templates (smaller squares), in accordance with the present disclosure. The two smaller squares with outlines are the two pressure templates that best match the data, and the hollow square in between those two outlined squares is the estimated position of the tourniquet.

FIG. 12 depicts an example of a screen used for calibrating the TMT virtual mentor to provide training on applying a CRoC, in accordance with an exemplary embodiment of the present disclosure.

FIG. 13 depicts an example of a pressure template calibration screen of a TMT virtual mentor, shown prior to calibration, in accordance with an exemplary embodiment of the present disclosure.

FIG. 14 depicts an example of a pressure template calibration screen of a TMT virtual mentor, shown after calibration is complete, in accordance with an exemplary embodiment of the present disclosure.

FIG. 15 depicts an example of a trainee feedback display for a TMT virtual mentor showing a correct application of a CRoC, in accordance with an exemplary embodiment of the present disclosure.

FIG. 16 depicts a trainee feedback display for a TMT virtual mentor showing a partial application of the AAJT, in accordance with an exemplary embodiment of the present disclosure.

FIG. 17 depicts a trainee feedback display for a TMT virtual mentor showing a partial application of AAJT but with too much applied pressure, in accordance with an exemplary embodiment of the present disclosure.

FIG. 18 depicts an ellipse fitting algorithm that can be used to identify the orientation of the tourniquet based on the pressure data, in accordance with exemplary embodiments of the present disclosure.

FIG. 19 depicts a sensor calibration process, giving users the ability to designate a line as the correct location in which to apply a tourniquet, in accordance with an exemplary embodiment of the present disclosure.

FIG. 20 depicts a sensor pad with a simulated anterior superior iliac spine (ASIS) landmark embedded in the fake tissue, in accordance with exemplary embodiments of the present disclosure.

FIG. 21 includes views (A) and (B), depicting exemplary sensor pad designs, in accordance with embodiments of the present disclosure.

FIG. 22 depicts an example of a screen for visualizing TMT data from training exercises, in accordance with an exemplary embodiment of the present disclosure.

FIG. 23 includes two views (A, B) showing an exemplary embodiment of an assessment module, in accordance with the present disclosure.

FIG. 24 depicts an assessment module linked with a sensor system, in accordance with an exemplary embodiment of the present disclosure.

FIG. 25 depicts another embodiment of a sensor system, in accordance with the present disclosure.

FIG. 26 depicts a pair of exemplary embodiments of a combined assessment module and live feedback display, in accordance with the present disclosure.

### DETAILED DESCRIPTION OF ILLUSTRATIVE EMBODIMENTS

Illustrative embodiments are now described. Other embodiments may be used in addition or instead. Details that may be apparent or unnecessary may be omitted to save space or for a more effective presentation. Some embodiments may be practiced with additional components or steps and/or without all of the components or steps that are described.

An aspect of the present disclosure provides or uses a combination of sensors and software as a tourniquet master training (“TMT”) system, process, and/or algorithm to enhance a process of learning how to apply a junctional tourniquet to non-compressible hemorrhages. A force sensing system is preferably employed, which provides detailed feedback on the physical process of how the tourniquet was or is being applied, and accordingly offering novel flexibility in application and sensing.

Embodiments of the present disclosure provide a sensor-based TMT system usable with a variety of manikins and application areas. Exemplary embodiments of the present disclosure include a TMT system providing scenario-based training featuring (1) a reconfigurable sensor system linked to a software-based virtual mentor (or tutor) that provides objective assessment during training, and (2) a mobile mentor (or, tutor) that provides refresher training. Because of the flexibility provided, TMT systems can be very affordable and also configurable for future advances, including new application areas (e.g., axillary applications) for both existing and future devices.

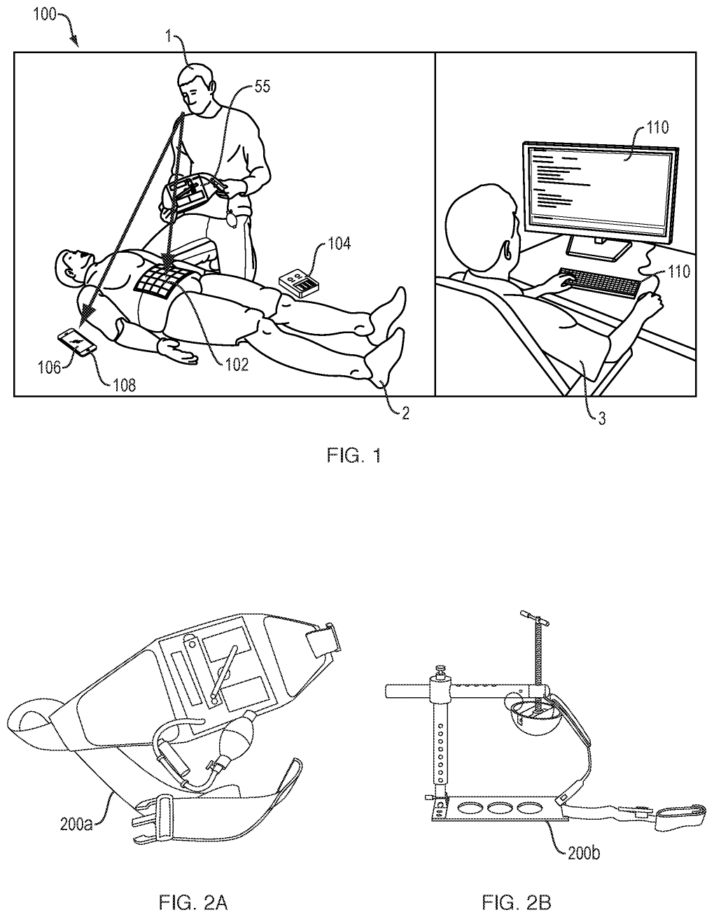

FIG. 1 depicts an example of a TMT system 100 in accordance with the present disclosure, which can be used for live training in the use of junctional tourniquet devices. TMT system 100 includes a sensor pad 102, an assessment module 104, a live feedback display 106, a mobile mentor (tutor) 108, an instructor assessment software no, and, optionally, supporting instructional materials (not shown). In the embodiment shown, the assessment module 104 is a wireless version; wired versions may be used in other embodiments or applications. Each component of the system 100 may optionally be housed in rigid or waterproof casings depending on the specific embodiment.

With continued reference to FIG. 1, at the left, a trainee applies 1 an AAJT (indicated as junction tourniquet “JT”) to a manikin 2 outfitted with the sensor pad 102 of the TMT system 100. In operation of the system 100, i.e., during training, the assessment module 104 (by the manikin's knee) reads and processes pressure data from the sensors of the sensor pad 102. Live feedback on the tourniquet's location and pressure is presented to the trainee 1 on a mobile device (near the manikin's arm), which in this embodiment includes the mobile mentor 108 and the live feedback display 106. At right, an instructor 3 can review the trainee's performance metrics on the instructor assessment software (indicated by keyboard no and display no), e.g., after the training session has concluded.

The sensor pad 102 detects the force applied by a tourniquet JT on a simulated wound and its signals are used by the assessment module 104 and/or instructor assessment software no to discriminate between different placements (e.g., on the manikin) of the tourniquet. Other optional data that the sensor pad 102 may additionally capture include the absolute position of the sensor system (such as through GPS) or temporal data. Exemplary versions of the sensor pad 102 may include additional anatomical representations such as simulated belly fat and/or bones or portions of bones (e.g., a sagittal crest), or other anatomical “landmarks.”

An exemplary embodiment of the sensor pad 102 includes a sheet of acrylonitrile butadiene styrene (“ABS”) plastic with a matrix of force-sensing sensors adhered to (positioned on) the top of it, with a layer of silicon rubber containing a marble adhered on top of that. In exemplary embodiments force-sensing sensors are preferably force-sensing resistors (“FSR”). In some embodiments, an Interlink Electronics FSR 406 model of force sensing resistor may be used for the force sensor(s). Another embodiment of the sensor pad includes a sheet of paper with multiple individual FSR sensors taped on top, with a layer of silicon rubber placed loosely on top of that. In another embodiment, four Sensitronics ShuntMode Matrix Array sensors are used, and these are encased on all sides by silicone rubber (i.e., they do not rest on a plastic surface). In yet another embodiment, two Kitronyx MP2508 10×16 FSR Matrix sensors are used, connected to a flexible printed circuit board (PCB), all of which are encased in the silicon rubber. The sensor pad 102 is preferably connected to the assessment module 104 in order to transmit its data to the assessment module 104. This connection can be one or more wires, or it can be a wireless mechanism, e.g., Bluetooth, XBee, or the like. In exemplary embodiments, a sensor pad 102 used individual 1.5″×1.5″ Interlink force sensing resistors adhered to a sheet of paper. In alternate embodiments, a sensor pad 102 can use a single printed pad containing multiple sensors inside of it for more precise measurements and reduced risk of damage.

The assessment module 104 preferably either stores all data produced by the sensor pad 102 or transmits the data to the instructor assessment software 110 so that it may store the data. One of either the assessment module 104 or the instructor assessment software 110 preferably computes metrics on the data coming from the sensor pad 102, depending on the specific embodiment. These metrics can include (but are not limited to) the elapsed time, the pressure exerted on the pad, and the center of mass of objects placed on top of the pad.

The assessment module 104 may optionally include a display, e.g., for indicating when data is being recorded and when pressure exerted on the pad is high enough to stop a simulated bleed. In one embodiment, the assessment module 104 is an Arduino Leonardo with an LCD screen, button matrix, SD disk writer, USB port, and power supply. Another embodiment of the Assessment Module is a commercial data acquisition device serving as an intermediary between the Sensor Pad and the instructor assessment software no. In another embodiment, the assessment module is a Samsung Galaxy Active Tab 2 with custom software deployed onto it. The assessment module 104 is preferably connected to the instructor assessment software no in order to transmit its data to the instructor assessment software no. This connection can be one or more wires, or it can be a wireless mechanism such as Bluetooth, XBee, or the like.

The instructor assessment software no preferably produces logs and summaries of the data and metrics. In an exemplary embodiment, the instructor assessment software 110 is a Java desktop application computing metrics, presenting data summaries, and producing logs.

The live feedback display 106 preferably visualizes the placement of objects on top of the sensor pad 102 and updates in real time. The live feedback display 106 preferably visualizes the location, amount of pressure exerted, and elapsed time. The live feedback display 106 may also visualize steps in the sequence of actions required to place the object, e.g., junctional tourniquet JT.

In exemplary embodiments, the live feedback display is or includes a mobile application on a mobile device such as a “cell” phone. In other embodiments, the live feedback display can be a window (e.g., a dedicated or separate window) within a user interface (e.g., display screen) used for or with the instructor assessment software no. The live feedback display 106 preferably either connects to the instructor assessment software 110 or to the assessment module 104, whichever is computing metrics on the placement of objects on the Sensor Pad. This connection can be either wired or wireless. The Mobile Mentor preferably presents didactic instruction, quizzes, and supporting media to train the placement of a specific object on the sensor pad. One specific embodiment of the Mobile Mentor is a mobile phone application with a sequence of screens providing instruction on how to place a junctional tourniquet.

The Supporting Instructional Materials (not shown) can provide the same type of content as the Mobile Mentor but may be present in a different format, e.g., including paper, and/or depth of detail or extent of subject matter coverage.

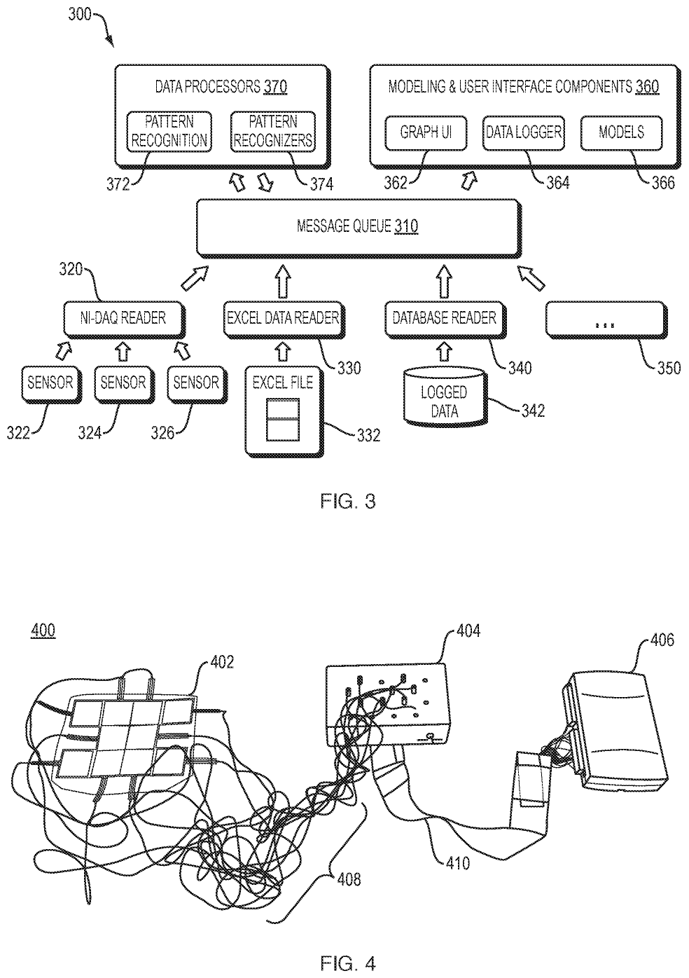

FIG. 3 depicts different junctional tourniquets, and includes views (A)-(B), with (A) showing an abdominal aortic junctional tourniquet (“AAJT”) 200A, and (B) showing a combat ready clamp (“CRoC”) 200B. In addition to the junctional tourniquets shown, other types of junctional tourniquets and junctional tourniquet devices may be used with the scope of the present disclosure.

FIG. 3 depicts a diagram of an example of an operational flow path or process 300 of a message queue 310 serving to coordinate and organize input data, messages, and/or files from multiple sources and allow such to be queried by and submitted to software applications, in accordance with an exemplary prototype a TMT system according to the present disclosure. As shown, message queue 310 can receive data from a DAQ reader 320, which can receive data from sensors 322-326, e.g., sensors of a sensor pad. Message queue 310 can also receive data from an Excel data reader 330, which may, e.g., read or access one or more Excel files 332. Message queue 310 can also receive data from a database reader 340, which may access or receive (or even write to) a database or store of logged data 342 or the like. Message queue can also access (including reading from and/or writing to) other types and sources of data, as indicated at 350. Message queue 310 can access or communicate with one or more processors 370, which can be used for a number of functions and/or tasks, e.g., to run or host pattern recognition software/programs 372 and/or pattern recognizers 374. Message queue 310 can be connected to and/or access modelling and user interface components 360, which can include, but are not limited to a graphical user interface 362, a data logger 364, and one or more models 366. Various components shown, e.g., message queue 310, data processors 370, and/or modeling and user interface components 360 may be resident in an assessment module and/or instructor assessment software in some embodiments of the present disclosure.

FIG. 4 depicts hardware for an embodiment of a TMT sensor setup or system 400, in accordance with an exemplary embodiment of the present disclosure. As shown as left, to FSR 406 force sensing resistors (pressure sensors) are adhered to a sheet of paper; (center) These sensors connect to an Arduino which is housed inside a black protective casing; (right) The Arduino sends data to a National Instruments Data Acquisition Device (DAQ).

In exemplary embodiments, a TMT sensor setup may include different numbers and configurations of resistors, e.g., 8 FSR 406 force sensing resistors; the resistors can send data to a green printed circuit board (PCB) via reinforced wires; the PCB may connects directly into a data acquisition unit, e.g., a National Instruments DAQ.

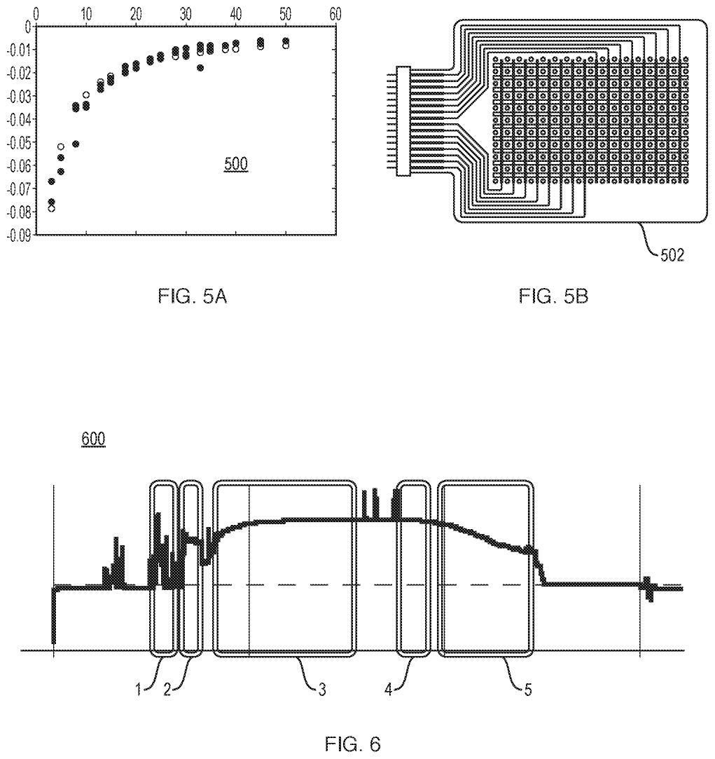

FIG. 5 depicts two view, at left curve 500 used to convert sensor voltages to force data (recorded or sensed voltage is indicated on the vertical axis, from top to bottom values of −0.01 to −0.09; x-axis in seconds, 0-60); due to voltage saturation, this curve was used to distinguish higher force values applied to the sensors. At right, a sensor produced by Sensitronics used as a component in an exemplary sensor pad is shown.

FIG. 6 depicts a plot 600 of sensor data recorded for AAJT application in one embodiment, with phases marked by human; from left to right: (1-2) buckling and tightening the belt, (3) twisting and fastening the windlass, (4) inflating the bladder, and (5) deflating the bladder. The data was normalized, with maximum force indicated as “1” (y-axis) and time from 0-90 seconds (x-axis).

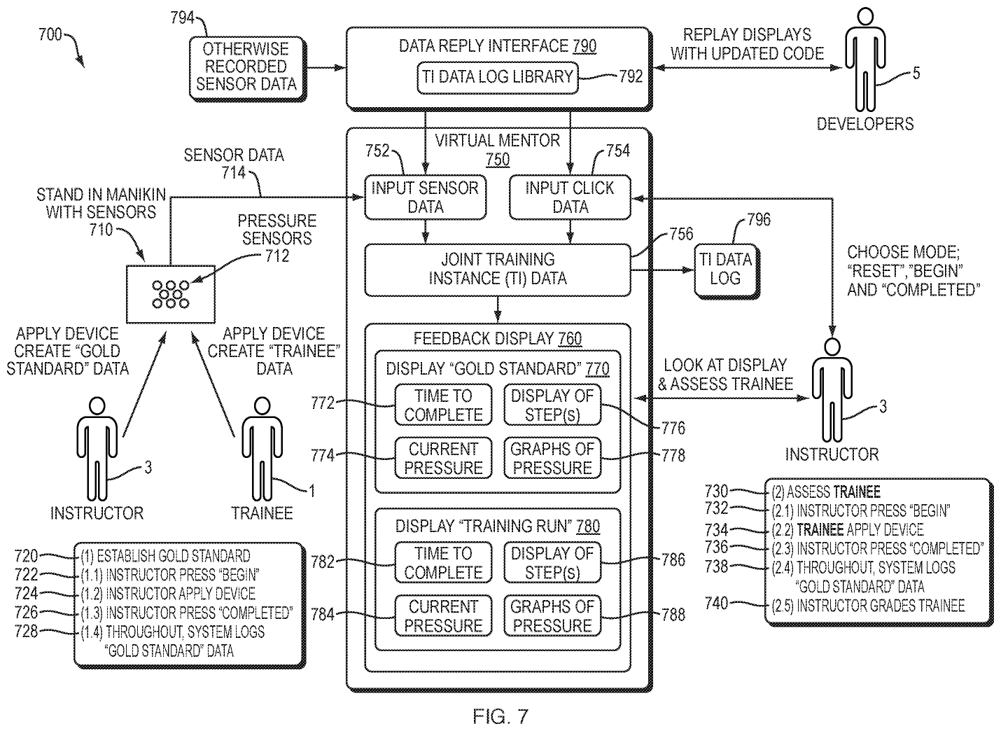

FIG. 7 depicts a training process or algorithm 700 for training of tourniquet application in accordance with exemplary embodiments of the present disclosure. A trainee 1 and instructor 3 are also shown. For the algorithm 700, a trainee or trainees will (does) try to match or exceed desired or targeted level of performance, e.g., performance attained previously or concurrently by an instructor or instructors. An example of such desired performance is indicated as “Gold Standard” in the figure. A stand-in manikin 710 with sensors (pressure or force sensors) 712 is shown at left, and serves to process pressure data and provide feedback. A virtual (software) mentor 750 is shown in the middle of the figure. At top, engineering displays provide additional information, e.g., to a development team.

Virtual mentor 750 can include a feedback display 760, and receive as inputs sensor data 752 and click data 754, as non-limiting examples, and these may be combined as joint training instance (“TI”) data 756. The TI data 756 may be logged or recorded by a TI data log 796. The virtual mentor 750 may receive other types of inputs in other embodiments of the present disclosure. The feedback display 760 can include or present a display related to the Gold standard 770 and/or a display of data from the trainee's actions, as indicated by “Trainee Run” 780. The Gold Standard display 770 can present time to complete 772, current pressure 774, display(s) of steps 776, and graphs of pressure 778. Similarly, the Training Run display 780 can present time to complete 782, current pressure 784, display(s) of steps 786, and graphs of pressure 788.

As shown at left, a gold standard performance can be established, as indicated at 720. For this, an instructor 3 can indicate a start of the procedure, e.g., by activating a user input mechanism such as pressing a “begin” button or switch or the like 722. The instructor 3 can then apply a given tourniquet device, e.g., an AAJT, CRoC, or the like to the stand-in manikin 710, as indicated at 724. When finished, the instructor 3 can then indicate completion of the tourniquet application 726, e.g., by activating a user input mechanism such as pressing a completed button, switch, or the like. The system, e.g., system 100 of FIG. 1, logs the data for the Gold Standard performance of the instructor 728. One or more trainees can accordingly be trained and assessed with the use of this Gold Standard performance.

With continued reference to FIG. 7, for such training and assessment, an instructor, e.g., instructor 3, can assess the performance of the trainee 2, as indicated at 730. For the assessment 730, the instructor can indicate the start or beginning of the assessment, e.g., by pressing a button or activating a switch, as indicated at 732. The trainee 1 then applies the junctional tourniquet device to the manikin 710, as indicated at 734. Throughout the process, the system records or logs the data received from the sensors 712 during the trainee's performance, as indicated at 738; the Gold standard data may be used for a comparison. The instructor 3 can then assess (e.g., grade) the trainee, as indicated at 740.

In exemplary embodiments, a participant setup screen can be provided by or for the TMT virtual mentor software; this screen can be used to collect information which allows performance data to be linked to specific users.

In exemplary embodiments, a trial setup screen can be provided by or for the TMT software; the instructor can indicate the type of tourniquet, the required pressure, trial, and the amount of time the trainee has to complete the application.

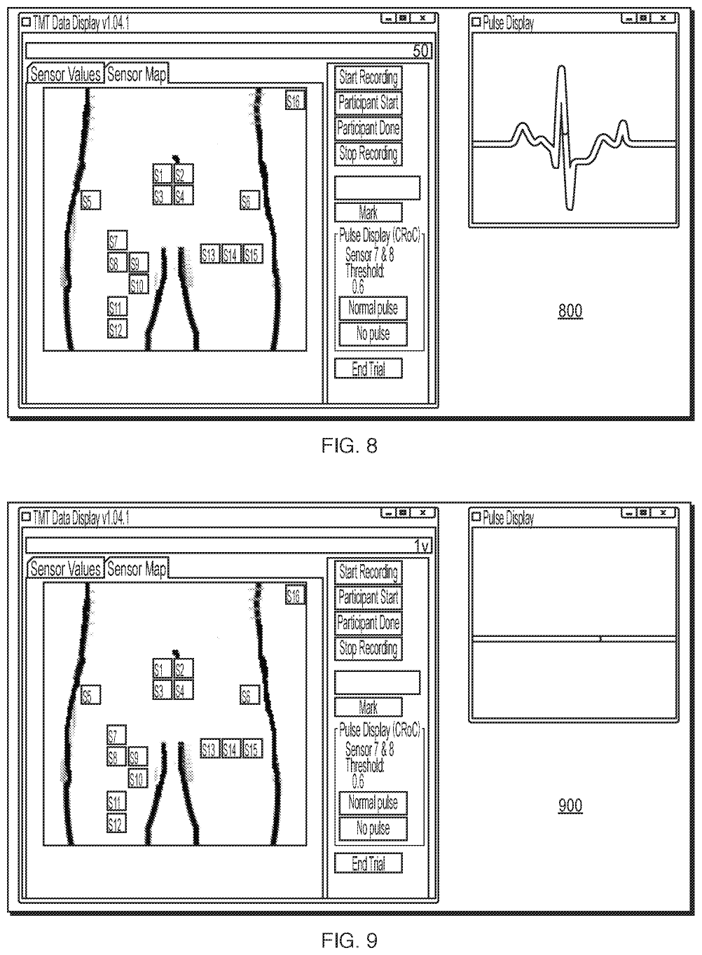

FIG. 8 depicts an example of a trainee display 800 for a version of a TMT virtual mentor according to the present disclosure. As shown, display 800 can provide feedback on three critical metrics: location, pressure, and time to complete.

In exemplary embodiments, an alternate feedback display can be provided by or for the TMT virtual mentor, which shows sensor data numerically rather than visually; this screen may be well-suited for use by developers.

FIG. 9 depicts an example of a trainee feedback display goo for a version of the TMT virtual mentor showing the outcome of a CRoC being applied to the correct location at the correct pressure; the pulse display at the right shows a flat line, indicating that blood flow has stopped.

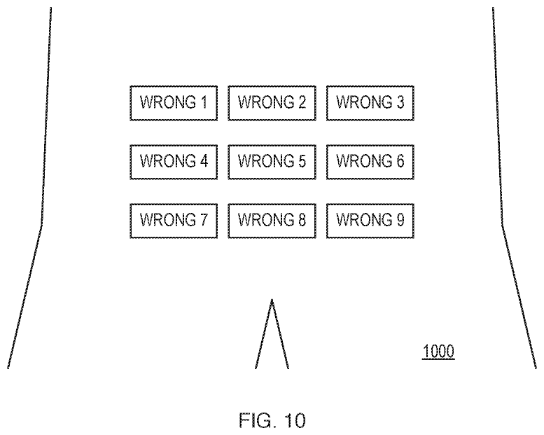

FIG. 10 depicts an application 1000 of nine pressure templates used to determine location metrics, e.g., on a stand-in manikin; each template corresponds to a tourniquet being at a particular location, and records what the sensor data look like when the tourniquet is at that location.

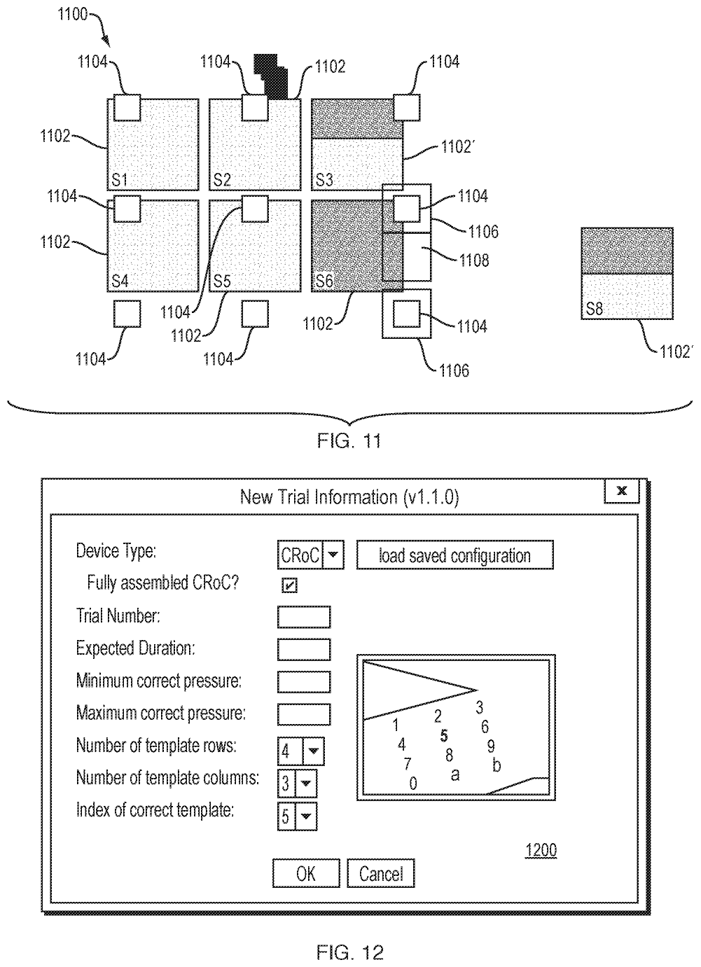

FIG. 11 depicts an example of an instructor display 1100 showing a number of sensors 1102 (larger shaded squares, some with split shading 1102′) and a number of pressure templates 1104 (smaller squares), in accordance with an exemplary embodiment of the present disclosure. The two smaller squares with outlines 1106 are the two pressure templates 1104 that best match the data, and the hollow square 1008 (in between those two outlined squares 1106) is the estimated position of the tourniquet.

FIG. 12 depicts screen 1200 for calibrating a TMT virtual mentor to be used for practicing applications of a CRoC, according to exemplary embodiments. As shown, a number and configuration of pressure templates can be specified, in this case, four (4) rows by three (3) columns for a total of 12 templates. The lightly-shaded “5” indicates that template 5 is centered on the correct location. The correct index can be selected by the instructor.

In alternate embodiments, a screen for calibrating an AAJT in a TMT virtual mentor, other numbers and configurations of pressure templates can be specified, e.g., 3 rows by 3 columns for a total of 9 templates, etc. The correct index can be selected by the instructor.

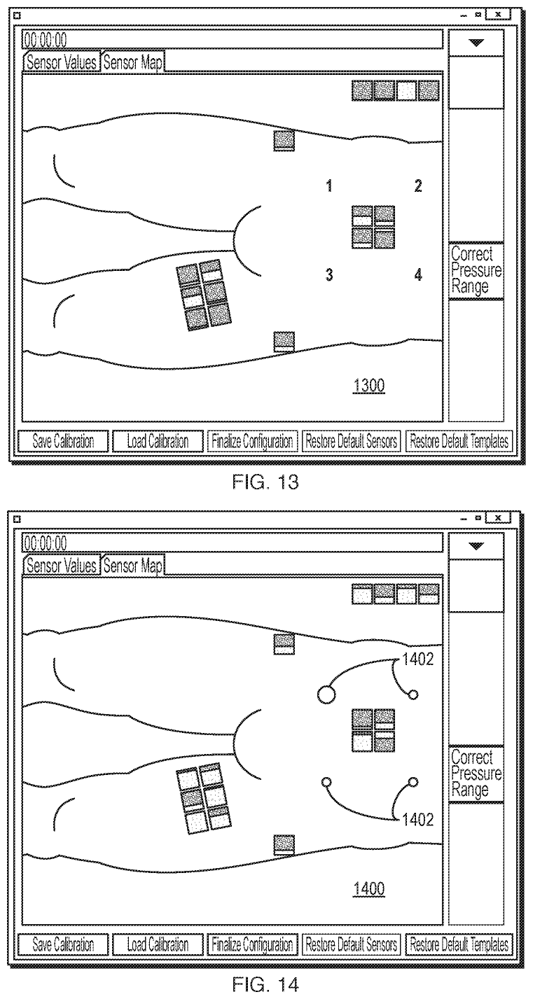

FIG. 13 depicts a pressure template calibration screen 1300 in an embodiment of a TMT virtual mentor, shown prior to calibration. In operation, an instructor would be shown the placement and numerical key for each Pressure Template. The instructor would then apply the AAJT in position 1, and then press/hit the ‘1’ key to record sensor data for that pressure template. This process would be continued for templates 2, 3, and 4.

FIG. 14 depicts a pressure template calibration screen 1400 in an embodiment of a TMT virtual mentor, shown after calibration is complete. The circles 1402 indicate that the instructor has created four complete Pressure Templates. At this point, the calibration file can be saved for future use. It can also be finalized which sends it to the feedback system for use during a training session.

In exemplary embodiments, an instructor control panel can be used for a TMT virtual mentor. Such a control panel can allow the instructor to control and/or indicate progression during the training exercise. For example, the instructor can indicate a trainee progressing through required trial tasks or steps by marking those tasks or steps off by pushing the buttons on the display. Examples of such trial tasks include, but are not limited to, “removed CRoC from bag,” “raised baseplate arm,” “Attached vertical arm,” “attached horizontal arm,” “Attached T handle,” “Attached disk,” “Positioned baseplate,” “Adjusted device arm(s),” “Positioned CRoC,” “Applied pressure,” “Buckled belt,” and “Tightened belt,” as non-limiting examples; other trial tasks may be indicated. That feedback can be passed to the trainee display.

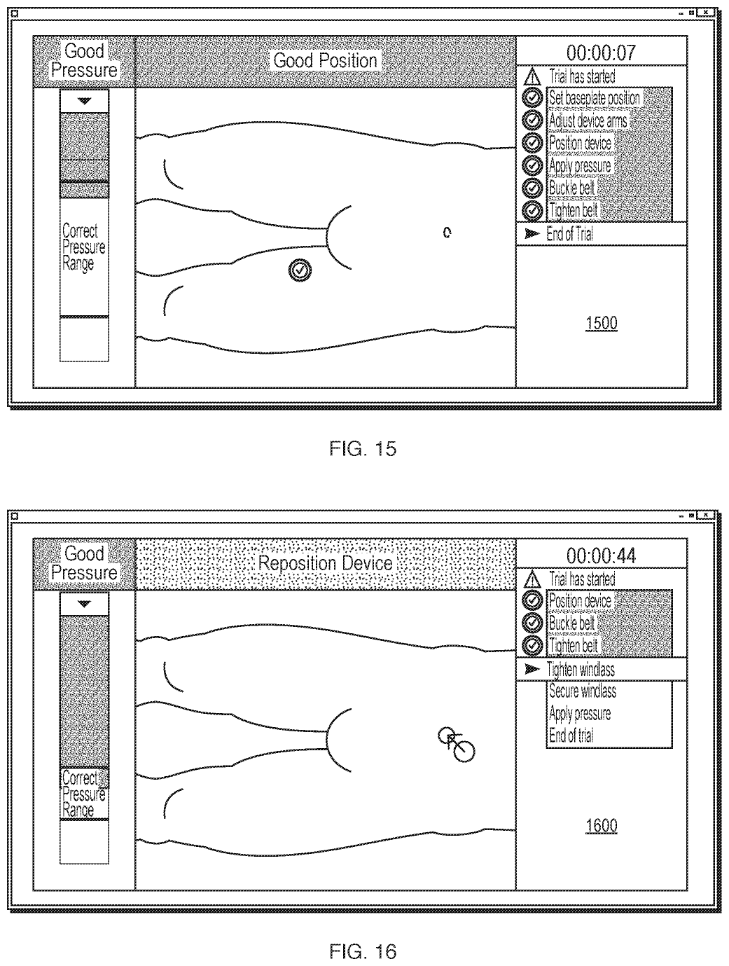

FIG. 15 depicts a trainee feedback display 1500 for an embodiment of a TMT virtual mentor, showing a correct application of the CRoC. Pressure feedback is shown at left. In this case, the pressure falls between the two black lines, and therefore is displayed in green with the heading “Good Pressure.” The position is shown in the middle (left to right), with a green check indicating the location is correct, and the green heading indicating the position is good. At the right, the steps have been correctly followed. The time is also shown in the top right (though in this case, it is a very small 7 seconds, which is unrealistic for a real application.)

FIG. 16 depicts trainee feedback display 1600 for an embodiment of a TMT virtual mentor, showing a partial application of the AAJT. Here, the pressure is shown as correct, but the range is different for the AAJT as the application area is different. The correct position is also different, since the AAJT must be centered on the belly button. Further, the Virtual Mentor has the location coded Yellow with the text “Reposition Device” because the position is close, but not exactly correct. The graphical display shows the direction the tourniquet needs to move. At right, the steps are being displayed. In this case, the first three steps have been completed, and the current step recommended is to “Tighten windlass.” The current time is 44 seconds, shown in the upper right. The system determines that repositioning is required using a software algorithm for determining the position of the tourniquet. This algorithm can be, e.g., a computation of center-of-mass, boundaries, or other similar properties, depending on the specific embodiment. The system uses another algorithm to compare the measured position of the tourniquet to a defined correct position. When center of mass is used as the position metric, the algorithm may measure the difference between the measured center of mass coordinate from the defined correct center of mass. For embodiments using other position metrics, different algorithms may be used, such as those that additionally consider the orientation of the device in addition to its absolute position. The need to reposition, length of time to complete an application or sub-task, improper applied pressure, and/or other performance characteristics measured during a training session can allow the system to make an inference as to the level of skill of the trainee, e.g., competent, not sufficiently competent, etc. Suitable statistical methods, e.g., implemented in or with the assessment module and/or instructor assessment software, can be used to facilitate or make such an inference.

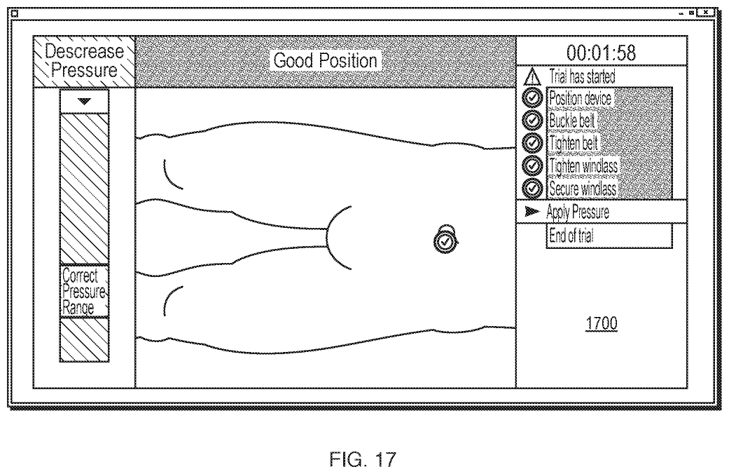

FIG. 17 depicts trainee feedback display 1700 for an embodiment of a TMT virtual mentor, showing a partial application of AAJT. The trainee has taken a few more steps, but is applying too much pressure. At left, the pressure bar is showing red, meaning too much pressure. The pressure bar exceeds the recommended level, and the heading is giving the feedback to “Decrease Pressure.” In the middle, the location is good. Note that the AAJT has a different location than the CRoC above, as the application area is different. Finally, at right, the trial is not complete, so the feedback is showing the current step is to apply pressure (meaning to inflate the bladder.) The participant has put too much pressure, and has to bleed some off. The current time is 1:58, shown in the upper right.

In exemplary embodiments, an after-action review (AAR) screen may be provided by a virtual mentor. This screen can be used to provide a summary of trainee performance after completing training. For example, portions of such a screen can provide information may be about the trainee and training session, a summary of performance on the critical metrics, and a detailed timeline of events during the session, as non-limiting examples.

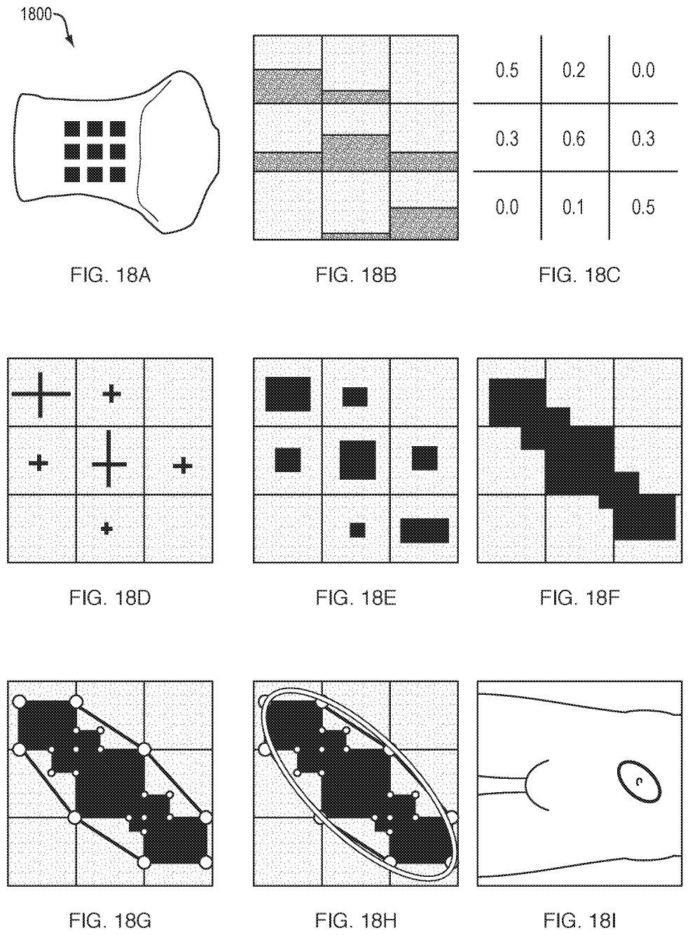

FIG. 18 depicts ellipse fitting algorithm 1800 that can be used to identify the orientation of the tourniquet based on the pressure data, in overview, in accordance with the present disclosure. View A shows the location of sensors on a manikin as setup in an embodiment of the system. View B graphically depicts the amount of pressure detected by each sensor for a particular training attempt, where squares with a greater amount of dark shading detect greater amounts of applied pressure. View C shows this same data numerically. ‘D’ shows for each sensor, the proportion of total pressure in each row and column that that sensor contributes, depicted as lines with different lengths. For instance, the centermost sensor contributes most of the total detected pressure in its vertical column as indicated by the large vertical line, and about half of the total detected pressure in its horizontal row, as indicated by the smaller horizontal line. ‘E’ shows how these lines are converted to fill rectangles, where the horizontal and vertical dimension of each rectangle correspond to the original dimensions of the lines (and thus to the proportion of total pressure in each row and column that is detected by individual sensors). ‘F’ shows the fill rectangles arranged against the edges of cells according to an algorithm that places rectangles against edges shared with neighbors that have similar detected pressure measurements. ‘G’ shows how a convex hull is computed from the arranged rectangles, where an outline is placed around all of the outermost points of all the rectangles. ‘H’ an algorithm that fits an ellipse to the points in the convex hull, thus giving an approximation of the shape of the object that led to the pressure detected by the sensors. ‘I’ shows the ellipse, drawn on the feedback display, indicating the estimated position of the device that was placed to apply pressure.

In exemplary embodiments, of a sensor configuration screen can be provided by a TMT virtual mentor. This can be used to allow instructors to define the placement of sensors on the manikin, including their relative position and orientation, their size, and the spacing between them. In exemplary embodiments, a setup page can be used for or provided by the TMT virtual mentor, which can show an overview of the configuration data currently being used.

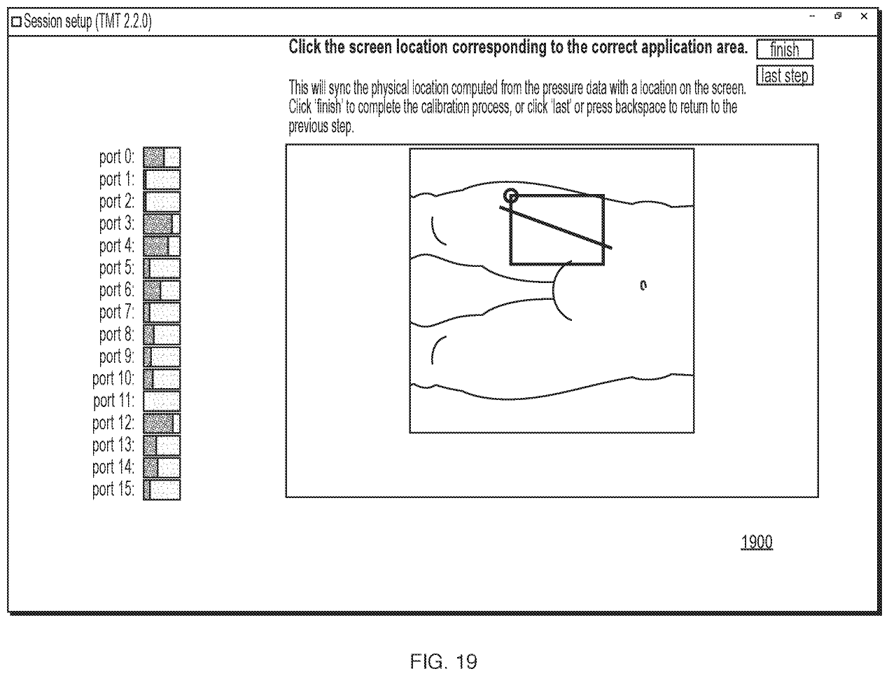

FIG. 19 depicts a sensor calibration process display 1900, which can give users the ability to designate a line as the correct location in which to apply the tourniquet (e.g., the straight line). It can also give users the ability to graphically assign the grid position and dimensions to a corresponding area on the feedback display (e.g., the rectangle), allowing for better scaling and more accurate visual feedback. In exemplary embodiments, an instructor screen can include a working button to recalibrate the system mid-trial.

In exemplary embodiments, a mobile mentor, e.g., an AAJT mobile mentor can include or provide quiz pages. For example, one or more quiz pages can include questions that require the trainee to select the appropriate location for applying the tourniquet, testing their decision-making skills. A quiz page may include one or more figures that serve to assess the trainee's declarative knowledge of the steps of applying a tourniquet, e.g., a AAJT, such as by requiring the trainee to identify a missing step. One or more figures may facilitate assessment of a trainee's procedural knowledge by requiring the trainee to place the steps in the correct order. An exemplary embodiment of the mobile mentor may be built using the “Declarative to Procedural 2.0” (D2P2) web tutoring framework, or similar frameworks such as GuideView or the Generalize Intelligent Framework for Tutoring (GIFT).

In exemplary embodiments, a high-level editor can be used for creating and organizing pages in the tutor (mobile mentor), e.g., a D2P2 high level editor. In exemplary embodiments, an authoring tool, e.g., a GuideView authoring tool, can be used to lay or design out tutors (or tutorials) in the form of branching flowcharts. In exemplary embodiments, a page at the beginning of an AAJT mobile mentor can provide information about the tutor or mobile mentor itself. In exemplary embodiments, a concepts and skills overview page can be used for or provided by the AAJT mobile mentor. In exemplary embodiments, a quiz page may ask a trainee to correctly match the name of a part of a tourniquet, e.g., a AAJT, to text (e.g., a letter or number) labeling that part. In exemplary embodiments, a virtual patient trauma simulation can be used to facilitate training. For example, a user can interact with a virtual patient by selecting various tools and actions to use on the patient. The user can also observe various physiology properties of the patient, such as by palpating or inspecting various body regions or by applying a pulse oximeter.

Training can include the use of wireframe of similar graphics. In exemplary embodiments, for example, two wireframes shown with junctional tourniquets can be displayed for a trainer to show how a junctional hemorrhage scenario could be implemented in a trauma simulation; this may facilitate the training of cognitive and decision-making skills for junctional tourniquets. For example, a user (trainee) would interpret physiological data and wound location to determine whether a junctional tourniquet is appropriate, which one to use, and where to place it. This scenario assesses their knowledge of the indications and contraindications for use of the various tourniquets. A could be tasked with selecting the correct steps for applying the AAJT, and perform them in the right order and in an appropriate amount of time. Such scenarios may be used to assess declarative knowledge about the steps and parts of the AAJT (or similar JT).

In exemplary embodiments, a Kitronyx Snowboard microprocessor may be used for an assessment module, e.g., 104 of FIG. 1. This microprocessor can be used to convert analog signals from the sensor pad into digital signals that can be used by the assessment module. In exemplary embodiments, Sensitronics' XactFSR technology can be used to create custom sensing matrices for a sensor pad, e.g., pad 102. In other embodiments, a non-customized Sensitronics ShuntMode sensor matrix was used. In exemplary embodiments, TMT sensors were a version of this matrix with custom sensor placement and sensitivities. In exemplary embodiments, a Snowboard development kit can be used for as assessment module. The Kit includes a Snowboard microcontroller, two different sizes and shapes of Kitronyx sensors, and an adaptor for connecting to sensors with a larger number of sensing units.

Exemplary embodiments were tested for sensor sensitivity, with an assessment being made of the changes in readings on the Sensitronics ShuntMode sensor as a result of additional presses on the hand pump, starting with an SJT that is fully tightened and secured over the sensors. In testing, it was shown that the sensors can clearly detect when the bottom of the SJT changes shape due to inflation (the SJT bladder includes a series of rings at the bottom which extend at different points depending on the amount of inflation).

In exemplary embodiments, instead of a breadboard, wires can be connected to both sensor interfaces on the Snowboard microcontroller. This is so that both interfaces can be observed when designing and building the enclosure. During use, each assessment module can preferably be connected to only a single sensor pad.

In exemplary embodiments of an assessment module and its electrical components, a standard SanDisk writer can be used for data logging. In other embodiments, a modified SanDisk writer can be used, which includes a GPS tracker and has the ability to log GPS coordinates.

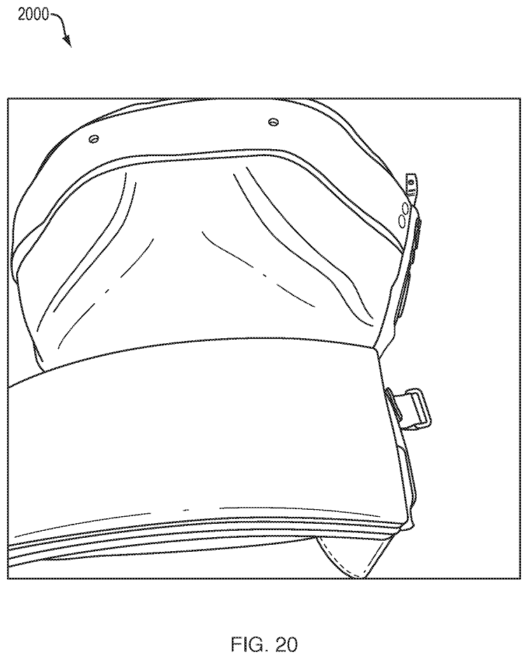

FIG. 20 depicts a sensor pad 2000 with a simulated anterior superior iliac spine (ASIS) landmark embedded in the fake tissue. Like the other sensor pads, the tissue layer for this pad is preferably made from Smooth-On products Eco-Flex and Slacker. In other embodiments, other materials may be used as well such as food coloring and baby powder. A one-inch diameter marble was placed in the mold before setting and embedded in the tissue to produce the landmark. This enables the sensor pad to represent relevant anatomy like the ASIS even when the pad is used on unrealistic anatomical representations like this back brace.

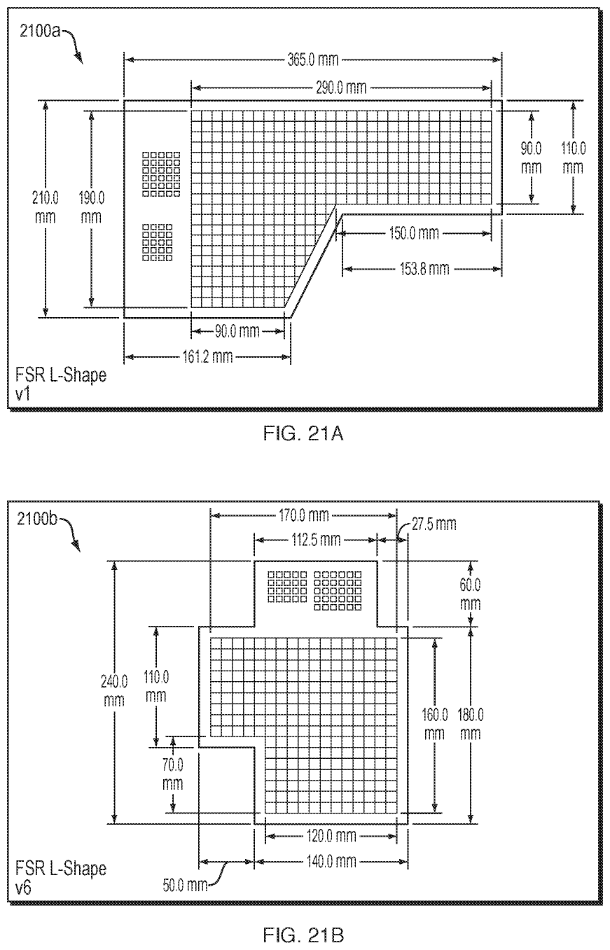

FIG. 21 includes views (A) and (B), depicting exemplary sensor pad designs 2100A and 2100B, in accordance with embodiments of the present disclosure; these are merely given as examples, and other configurations and sizes of sensor pads are of course possible within the scope of the present disclosure.

In exemplary embodiments, a Hammond Arduino project enclosure can be used and modified to enclose an assessment module. This is a COTS enclosure from Hammond Manufacturing. This rugged enclosure best matches the dimensions of the Assessment Module parts when they are arranged compactly. This enclosure can be modified simply with a power drill in order to add holes for the buttons and screen and for the ports for the sensors, USB port, and battery pack.

In exemplary embodiments, one or more interaction-level “Exercises and Data” screen can be used in the instructor assessment station desktop software application. This screen can allow the instructor to view data from a particular exercise or group of trainees. When the instructor selects a particular exercise or listed group of trainees, the “View Data” button can be activated. For further example, when the “View Data” button is clicked, a new window can be caused to appear prompting the instructor for the type of data they'd like to view. In this case, they can view background info, raw sensor data, and timelines of training events for a particular exercise or group of trainees. As another example of an interaction-level screen, an “Add Record” window can be provided within the “Individual Trainees” screen in the Instructor Assessment Station software application. This screen can allow instructors to add new trainees and fill in data for that new trainee by entering text in a set of labeled text boxes. Interaction-level screens or windows can be used for the “Add Record” window within the “Individual Trainees” screen in the Instructor Assessment Station software application. Using such a screen or window, instructors can modify the data structure using the “Add Field” button, which populates records with an entirely new field. In exemplary embodiments, a “Home screen” for the instructor assessment station software, instructors may be provided access to data on their trainees, classes, and TMT devices. Instructors may be allowed to and provided with the ability to define fields for information they consider relevant to store about their trainees. A search bar can be used to allow them to filter records based criteria within the fields. A window can be provided for editing individual trainee data or creating new records for trainees.

In exemplary embodiments, a window for viewing data on groups that the instructors are responsible for. Groups can be organized according to the instructor's preference, so they can represent individual classes, military units, etc. In exemplary embodiments, a window can be provided for viewing and editing metadata on groups. This data includes individuals that are members of the group, exercises that the group has participated, and other descriptive information such as the group's leader, location, and description. As with the fields in the records for individual trainees, instructors have the freedom to define fields that they think are relevant.

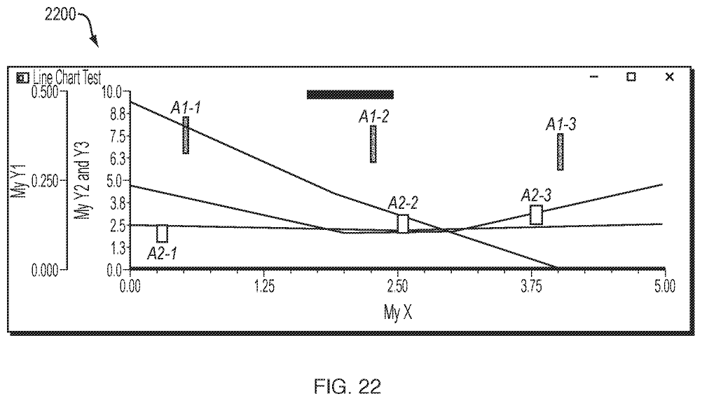

FIG. 22 depicts a screen for visualizing TMT data from exercises. The display can visualize multiple simultaneous line plots on shared or separate axes and can also display other annotations. This can be used to visualize data from individual sensors, aggregated metrics, and specific training events logged by the system (e.g. “exercise ended”, or “AAJT placement completed”). In this example, the screen is displaying dummy data.

In exemplary embodiments, an assessment module can includes a touch screen rather than text screen. Users can select to begin training or to manage the data that is logged during training. In exemplary embodiments, an assessment module includes a configuration process prior to training in which the instructor can set several parameters about training. This screen lets them select which tourniquet will be used with the system. In exemplary embodiments, a set of screens from an assessment module configuration process can allow instructors to set the thresholds for the minimum amount of pressure to stop bleeding and the maximum amount of pressure beyond which the tourniquet will cause additional injury. In exemplary embodiments, a screen from the configuration process for an assessment module can allow instructors to set the difficulty level of the training. For example, an easy scenario may reach a steady state once the tourniquet is applied, while a difficult scenario may cause “re-bleeding” to simulate vascular movement, or as a result of air transport. In exemplary embodiments, a data management screen of an assessment module can allow instructors to either delete data files or transfer them to a computer.

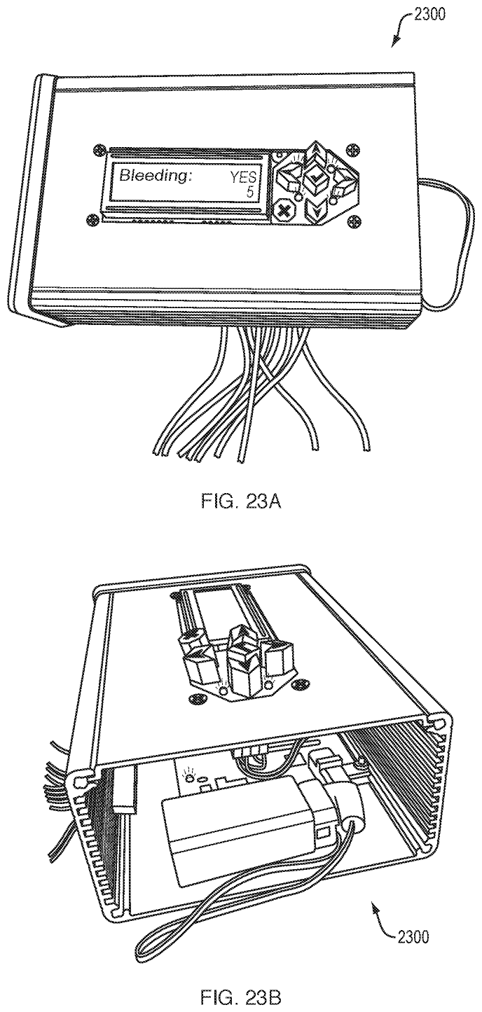

FIG. 23 includes two view (A, B) showing an exemplary embodiment of an assessment module 2300, in accordance with the present disclosure. View A depicts a completed assessment module with rugged enclosure and feedback screen. View B depicts a view inside the enclosure of the assessment module, showing a partial layout of the electronics.

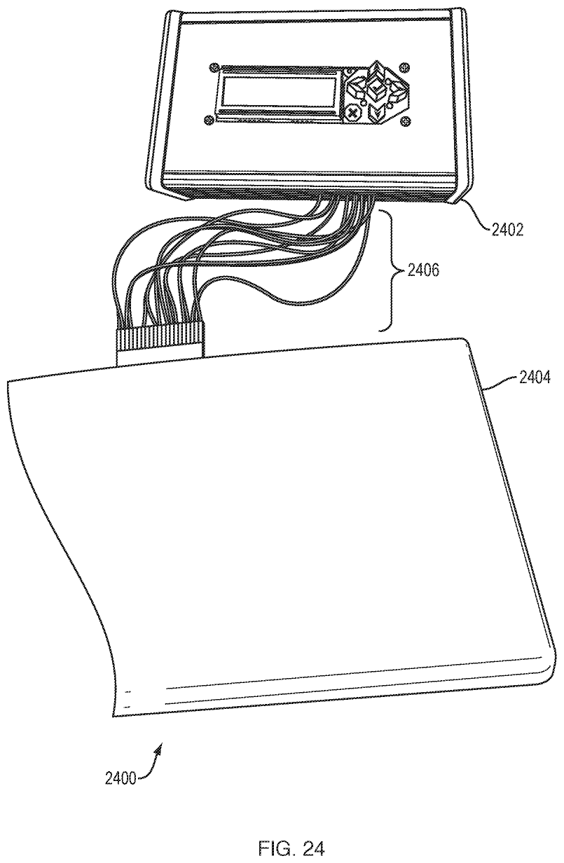

FIG. 24 depicts an assessment module 2402 linked with a sensor system (sensor pad) 2404 by wires 2406, in accordance with an exemplary embodiment 2400 of the present disclosure. The white opaque material at the bottom is the sensor system, wired to the blue device which is the ruggedized assessment module.

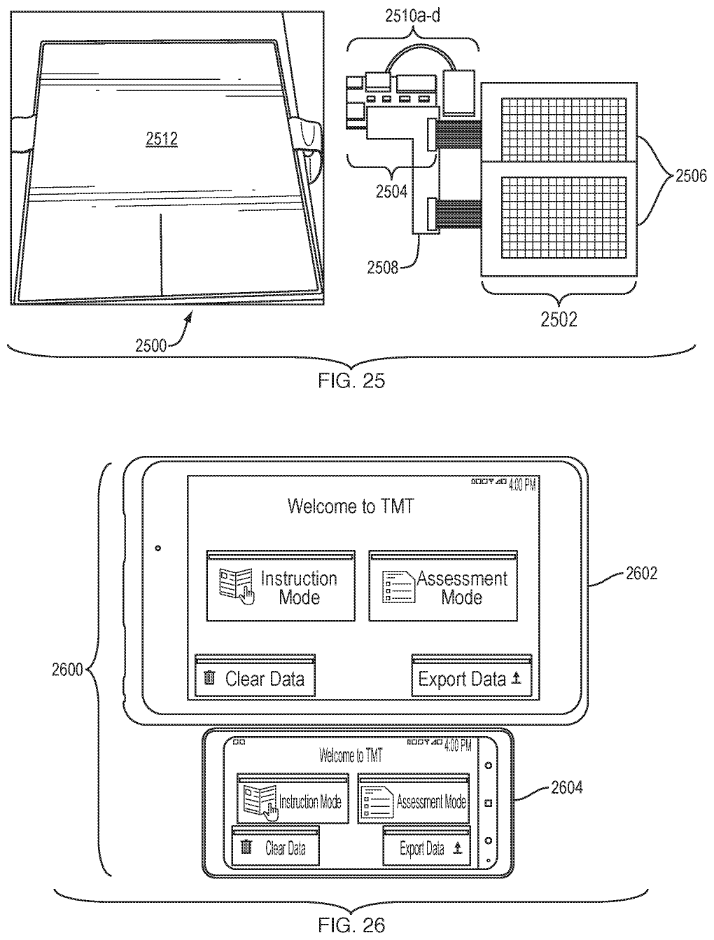

FIG. 25 depicts another embodiment of a sensor system 2500, in accordance with the present disclosure. At right is shown the sensor pad 2502, while at left are shown the electrical components 2504 that give it its functionality; system 2500 includes two Kitronyx FSR Matrix sensors 2502 (black), a flexible printed circuit board 2508, and multiple attached components 2510a-d including a battery, power switch, GPS tracker, and Bluetooth device. The electrical components 2504 of the sensor pad are encased in a silicon rubber mix 2512 which has the texture of skin, enabling realistic tactile feedback of tourniquet placement. The silicon rubber also further protects the electrical components and conceals the location of the sensor pad so as not to give away the correct position to trainees. Any suitable GPS (including GLONASS) sensor or tracker may be used.

FIG. 26 depicts a pair 2600 of exemplary embodiments of a combined assessment module and live feedback display, in accordance with the present disclosure. In these embodiments, a Samsung Galaxy tablet (top) 2602 and phone (bottom) 2604 are used to house both of the assessment module and live feedback display, with selection icons for the two modes shown. Custom software deployed to these devices carried out both functions.

Accordingly, relative to previous techniques, embodiments of the present disclosure afford benefits and/or key distinguishing features, including (but not limited to) metrics on placement of a tourniquet over its target location is computed automatically and objectively (e.g. with respect to pre-defined metrics)—feedback on placement is then available to be presented in real time or another time. Training systems, methods, and components such as described above for embodiments of the present disclosure, which can be used to teach and refresh skills for the described types hemorrhaging injuries (in particular, non-compressible hemorrhages in junctional or inguinal areas), may be particularly useful or vital because such injuries are rare on the battlefield and difficult to train for.

In preferred embodiments, embodiments can utilize force sensing technology as described, i.e. force sensing resistors. In other embodiments, other force sensing technology may be used, e.g., capacitive touch sensors, inductive touch sensors, or the like.

In some exemplary embodiments, different specific physical prototypes have used the following components; these are merely given by way of examples and other suitable components may be used within the scope of the present disclosure:

Sensor Pad: Interlink FSR 406, Sensitronics ShuntMode array, Sensitronics ThruMode array, Kitronyx matrix sensor 1007, Kitronyx matrix sensor 1610, Smooth-on Ecoflex 00-30, Smooth-on Slacker, generic marbles, Digikey jumper cables, TAP Plastics ABS sheet;

Assessment module: Kitronyx Snowboard 1, Adafruit LCD displays (various models), Adafruit button matrix (various models), Crystalfontz LCD displays (various models), Sparkfun GPS shield, Sparkfun SD shield, Digikey battery clips, batteries (various brands), National Instruments NI-USB 6210, C++;

Live feedback display: JAVA, Apache ActiveMQ, Python, C++;

Instructor Assessment Software: C++; and

Mobile Mentor: Penn D2P2.

Unless otherwise indicated, the systems and components that have been discussed herein, e.g., shown and described for FIGS. 1, 3, and 7, are, or can be, implemented with, or include a specially-configured computer system specifically configured to perform the functions that have been described herein for the system or component. Each computer system includes one or more processors, tangible memories (e.g., random access memories (RAMs), read-only memories (ROMs), and/or programmable read only memories (PROMS)), tangible storage devices (e.g., hard disk drives, CD/DVD drives, and/or flash memories), system buses, video processing components, network communication components, input/output ports, and/or user interface devices (e.g., keyboards, pointing devices, displays, microphones, sound reproduction systems, and/or touch screens).

Each computer system may be a desktop computer or a portable computer, such as a laptop computer, a notebook computer, a tablet computer, a PDA, a smartphone, or part of a larger system, such a vehicle, appliance, and/or telephone system.

A single computer system may be shared by the systems and components that have been discussed herein, e.g., shown and described for FIGS. 1, 3, and 7. Each computer system may include one or more computers at the same or different locations. When at different locations, the computers may be configured to communicate with one another through a wired and/or wireless network communication system.

Each computer system may include software (e.g., one or more operating systems, device drivers, application programs, and/or communication programs). When software is included, the software includes programming instructions and may include associated data and libraries. When included, the programming instructions are configured to implement one or more algorithms that implement one or more of the functions of the computer system, as recited herein. The description of each function that is performed by each computer system also constitutes a description of the algorithm(s) that performs that function.

The software may be stored on or in one or more non-transitory, tangible storage devices, such as one or more hard disk drives, CDs, DVDs, and/or flash memories. The software may be in source code and/or object code format. Associated data may be stored in any type of volatile and/or non-volatile memory. The software may be loaded into a non-transitory memory and executed by one or more processors.

The components, steps, features, objects, benefits, and advantages that have been discussed are merely illustrative. None of them, or the discussions relating to them, are intended to limit the scope of protection in any way. Numerous other embodiments are also contemplated. These include embodiments that have fewer, additional, and/or different components, steps, features, objects, benefits, and/or advantages. These also include embodiments in which the components and/or steps are arranged and/or ordered differently.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain. Further, use of the terms “preferable” or “preferably” does not mean that something is absolutely necessary or required.

All articles, patents, patent applications, and other publications that have been cited in this disclosure are incorporated herein by reference.

The phrase “means for” when used in a claim is intended to and should be interpreted to embrace the corresponding structures and materials that have been described and their equivalents. Similarly, the phrase “step for” when used in a claim is intended to and should be interpreted to embrace the corresponding acts that have been described and their equivalents. The absence of these phrases from a claim means that the claim is not intended to and should not be interpreted to be limited to these corresponding structures, materials, or acts, or to their equivalents.

The scope of protection is limited solely by the claims that now follow. That scope is intended and should be interpreted to be as broad as is consistent with the ordinary meaning of the language that is used in the claims when interpreted in light of this specification and the prosecution history that follows, except where specific meanings have been set forth, and to encompass all structural and functional equivalents.

Relational terms such as “first” and “second” and the like may be used solely to distinguish one entity or action from another, without necessarily requiring or implying any actual relationship or order between them. The terms “comprises,” “comprising,” and any other variation thereof when used in connection with a list of elements in the specification or claims are intended to indicate that the list is not exclusive and that other elements may be included. Similarly, an element proceeded by “a” or “an” does not, without further constraints, preclude the existence of additional elements of the identical type.

None of the claims are intended to embrace subject matter that fails to satisfy the requirement of Sections 101, 102, or 103 of the Patent Act, nor should they be interpreted in such a way. Except as just stated in this paragraph, nothing that has been stated or illustrated is intended or should be interpreted to cause a dedication of any component, step, feature, object, benefit, advantage, or equivalent to the public, regardless of whether it is or is not recited in the claims.

The abstract is provided to help the reader quickly ascertain the nature of the technical disclosure. It is submitted with the understanding that it will not be used to interpret or limit the scope or meaning of the claims. In addition, various features in the foregoing detailed description are grouped together in various embodiments to streamline the disclosure. This method of disclosure should not be interpreted as requiring claimed embodiments to require more features than are expressly recited in each claim. Rather, as the following claims reflect, inventive subject matter lies in less than all features of a single disclosed embodiment. Thus, the following claims are hereby incorporated into the detailed description, with each claim standing on its own as separately claimed subject matter.

What is claimed is:

1. A tourniquet training system which can be placed on a manikin or other anatomical representation to detect spatial variations in pressure application, the system comprising:
a processor;
a sensor pad having a plurality of force-sensing sensors configured in a matrix;
an assessment module configured to read and process pressure data received from the sensors;
a feedback display in communication with the assessment module and operative to display feedback to a trainee participating in a training procedure applying pressure to the sensor pad;
a memory in communication with the processor via a communication infrastructure and storing computer-readable instructions that, when executed by the processor, cause the processor to:
(i) record force measurement data received from the sensors while an pressure-applying device is applied to the sensors;
(ii) provide feedback to the feedback display for the trainee.

2. The system of claim 1, wherein the sensor pad includes anatomical landmarks representing features of human anatomy.

3. The system of claim 1, further comprising an enclosure housing the assessment module.

4. The system of claim 1, wherein for presenting real-time feedback to the trainee based on the data obtained from the sensor pad.

5. The system of claim 1, wherein the processor is further configured to interpret data obtained from the sensor pad and to make an inference about the skill of the trainee at controlling a hemorrhage using a junctional tourniquet device.

6. The system of claim 5, further comprising a display for visualizing skill assessments of the trainee.

7. The system of claim 2, wherein the landmarks include a simulated anterior superior iliac spine (ASIS) landmark.

8. The system of claim 1, wherein the processor is configured to determine a position in space.

9. The system of claim 8, further comprising a GPS sensor configured to determine a position of the sensor received from a GPS system.
